(19) United States
(12) Patent Application Publication
(10) Pub. No.: US 2001/0053374 A1
(43) Pub. Date: Dec. 20, 2001

(54) Clear Microemulsions For Use In Hair Conditioner Formulations

(76) Inventors: Damon M Dalrymple, Columbus, OH (US); Monna Manning, Columbus, OH (US)

Correspondence Address: Leopold Presser ESQ, Scully Scott Murphy & Presser, 400 Garden City Plaza, Garden City, NY, 11530, US

(21) Appl. No.: 09/497,462

(22) Filed: Feb. 3, 2000

(51) Int. Cl. A61K 7/00; A61K 7/06; A61K 6/00

(52) U.S. Cl. 424/401; 424/70.1

(57) ABSTRACT

A microemulsion for use in personnel care products, home care products and fabric care products which is clear and has particle sizes that are less than the wavelength of light, said microemulsion comprising, as essential components; (a) a substantially water insoluble quaternary ammonium salt containing at least one long-chain aliphatic C₈₋₂₄ hydrocarbon group; (b) an oil or hydrophobic organic compound that is not dispersible in water on its own; and (c) a dispersant capable of preventing component (b) from coalescing into particle sizes that are above the wavelength of light, wherein the weight ratio of component (a) to component (b) to component (c) is from 2:0.5:0.4 to about 0.5:2:0.8. A clear, indefinitely dilutable microemulsion is obtained by quenching the same in water.

### DESCRIPTION

1. Field of the Invention

The present invention relates to a clear microemulsion formulation having average particle sizes that are less than the wavelength of light. The formulation remains clear even after dilution. Moreover, the microemulsions of the present invention are freeze/thaw stable; therefore, these formulations do not convert to a typical oil-in-water emulsion, i.e., cream, after being frozen. The microemulsions of the present invention are useful as a component in personal care products.

2. Background of the Invention

Quaternary ammonium salts such as dicetyl dimethyl ammonium chloride have been used in the prior art as one of the effective components present in hair conditioner formulations. The use of such quaternary ammonium salts minimizes the breaking, entanglement, static charge and difficulties associated in the combing of washed hair. In addition to these properties, a hair conditioner formulation should also impart softness, smoothness and an antistatic property to washed hair. If only a quaternary ammonium compound is employed, as in some prior art formulations, the required softness and smoothness is not obtained.

Accordingly, in order to obviate this deficiency, oils and/or fats such as higher alcohols, glycerides and paraffins have been incorporated into hair conditioner formulations of the prior art. A major commercial drawback with the incorporation of oils and fats into hair conditioner formulations, however, is that such formulations provide an opaque or pearlescent appearance to the formulation.

The function of a transparent liquid formulation when such oils and fats are employed, would provide a resultant hair conditioner formulation which would have an enhanced appearance and a high commercial value. Various attempts have been carried out in the prior art to achieve this goal. One of the most popular methods developed to date comprises incorporating an organic solvent such as ethyl alcohol, propyl alcohol, ethylene glycol or propylene glycol in an amount as large as 60 weight % or more into the formulation. The use of organic solvents in hair conditioner formulations helps solubilize the oil and fat component and aids in rendering the composition transparent. This approach also renders the conditioner agent more easily rinsed and not deposited on the hair.

Another known method to solubilize fats and oils is to employ a large amount of a highly hydrophilic non-ionic surfactant which is obtained by adding 20 to 40 moles of ethylene oxide to a higher aliphatic alcohol. This approach also does not deposit adequate conditioning agents on the hair. Moreover, there has been purposed a hair conditioner formulation which is free of oils and fats which employs the use of an aqueous solution containing a water-soluble cationic surfactant having one of the following formulas:

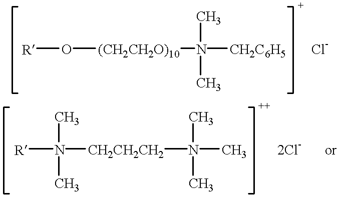

wherein R′ is an alkyl having 16 to 18 carbon atoms.

Transparent hair conditioner formulations formed by solubilizing cationic quaternary ammonium compounds, oils and fats using large quantities of organic solvents are disadvantageous because of their very high manufacturing cost and poor efficacy. Furthermore, methods that use large quantities of highly hydrophilic non-ionic surfactants suffer from the disadvantage that it is difficult to maintain the resulting transparent solution system in a stable condition (over various temperature changes such as freeze/thawing), causing a drastic reduction in the desired rinsing effect. When aqueous solutions of a water-soluble cationic surfactant are used in the absence of an oil, the product does not have a satisfactory rinsing effect.

In addition to the above mentioned problems, prior art hair conditioner formulations do not remain transparent upon dilution. Instead, upon dilution in water, prior art formulations generally have an opaque or pearlescent appearance. Moreover, prior art transparent hair conditioner formulations are not freeze/thaw stable. That is, upon freezing, the prior art hair conditioner formulations typically convert to an oil-in-water emulsion, i.e., these formulations become creamy in appearance. These two properties greatly limit the use of prior art microemulsions in hair conditioner formulations.

In view of the drawback characteristic of prior art transparent hair conditioner formulations, there is a continued need for developing an improved transparent microemulsion formulation which can be manufactured at low cost, yet is capable of maintaining a desired softness, smoothness and rinsing effect. In addition to the aforementioned properties, the improved microemulsion formulation should also minimize the breaking, entanglement, static charging and difficulties associated with combing washed hair.

### SUMMARY OF THE INVENTION

The present invention is directed towards novel microemulsions that are particularly suitable for use in personal care products. More specifically, the present invention is directed to microemulsions which are highly suitable for use in hair conditioner formulations.

The microemulsions of the present invention are clear, have average particle sizes that are less than the wavelength of light (average of 100 nm or less), are freeze/thaw stable and are easy to manufacture. More particularly, the microemulsions of the present invention remain clear even after substantial dilution in water (i.e., less than 99%).

This characteristic of a clear, substantially dilutable microemulsion is quite surprising since prior art microemulsions typically become cloudy (opaque) upon further dilution in water. To obtain the clear dilutable microemulsion of the present invention, applicants have determined a unique and appropriate combination of components and their concentrations necessary for achieving the clear, dilutable microemulsion of the present invention. Moreover, applicants have also discovered a methodology that when practiced unexpectedly provides the clear, substantially dilutable microemulsion of the present invention.

One aspect of the present invention relates to a microemulsion which is clear and has average particle sizes that are less than the wavelength of light, said microemulsion comprising, as essential components:

(a) a substantially water insoluble quaternary ammonium salt containing at least one long-chain aliphatic C₈₋₂₄ hydrocarbon group;

(b) an oil or hydrophobic organic compound that is not dispersible in water on its own; and

(c) a dispersant capable of preventing component (b) from coalescing into particle sizes that are above the wavelength of light, wherein the weight ratio of component (a) to component (b) to component (c) is from about 2:0.5:0.4 to about 0.5:2:0.8.

Another aspect of the present invention is directed to a substantially dilutable microemulsion which remains clear after the addition of water. The infinitely dilutable microemulsion of the present invention comprises, as essential components:

(a) a substantially water insoluble quaternary ammonium salt containing at least one long-chain aliphatic C₈₋₂₄ hydrocarbon group;

(b) an oil or hydrophobic organic compound that is not dispersible in water on its own;

(c) a dispersant capable of preventing component (b) from coalescing into particle sizes that are above the wavelength of light; and

(d) at least 50% water, wherein the weight ratio of component (a) to component (b) to component (c) is from about 2:0.5:0.4 to about 0.5:2:0.8.

A further aspect of the present invention relates to a method of preparing the clear dilutable microemulsion of the present invention. Specifically, the method of the present invention comprises the steps of:

(i) providing a clear microemulsion, said clear microemulsion comprising (a) a substantially water insoluble quaternary ammonium salt containing at least one long-chain aliphatic C₈₋₂₄ hydrocarbon group; (b) an oil or hydrophobic organic compound that is not dispersible in water on its own; and (c) a dispersant capable of preventing component (b) from coalescing into particle sizes that are above the wavelength of light, wherein the weight ratio of component (a) to component (b) to component (c) is from about 2:0.5:0.4 to about 0.5:2:0.8; and

(ii) quenching the clear microemulsion by adding a sufficient amount of water to provide a clear, diluted microemulsion.

### BRIEF DESCRIPTION OF THE DRAWINGS

FIG. 1 is a ternary phase diagram of a microemulsion of the present invention showing the clear regions of a concentrated (solid line) and 10% diluted (dotted line) formulation.

FIG. 2 is a ternary phase diagram of another microemulsion of the present invention illustrating the clear regions of a concentrated (solid line) and 10% diluted (dotted line) formulation.

FIG. 3 is a ternary phase diagram of yet another microemulsion of the present invention showing the clear regions of a concentrated (solid line) and 10% diluted (dotted line) formulation.

FIG. 4 is a graph of % transmittance at 460 nm vs. ml water added for a microemulsion in which water is added in small increments (solid line) and a microemulsion in which all the water is added at once (dotted line).

### DETAILED DESCRIPTION OF THE INVENTION

The present invention provides a novel microemulsion formulation which is clear even after dilution in water; the microemulsion is freeze/thaw stable. Moreover, the microemulsion of the present invention is characterized by particle sizes (average) that are less than the wavelength of light. Such microemulsions having the above properties are particularly useful as a component in hair conditioner formulations or in other personal care products.

The first component of the microemulsion of the present invention is a substantially water insoluble quaternary ammonium salt containing at least one long-chain aliphatic C₈₋₂₄ hydrocarbon group. The quaternary ammonium compound is used in the present formulation as an emulsifier. The term “substantially water insoluble” when used in conjunction with the quaternary ammonium compound denotes a quaternary ammonium compound in which the solubility in water is extremely low (less than about 5 mg/ml). In other words, the quaternary ammonium compounds employed in the present invention are incapable of forming a 5% aqueous solution.

Suitable quaternary ammonium compounds that are substantially water insoluble and thus can be employed in the present invention include those compounds having one of the following formulas:

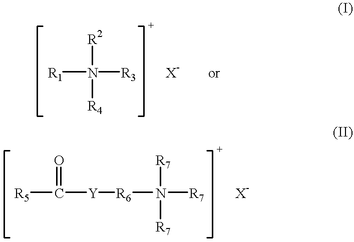

wherein R₁ is a long-chain aliphatic hydrocarbon having from 8 to 24 carbon atoms; R₂ is a long-chain aliphatic hydrocarbon having from 8 to 24 carbon atoms, an alkyl group having from 1 to 6 carbon atoms or a hydroxyalkyl having from 1 to 6 carbon atoms; R₃ is hydrogen, benzyl, an alkyl having from 1 to 6 carbon atoms or hydroxyalkyl having from 1 to 6 carbon atoms; R₄ is an alkyl having from 1 to 6 carbon atoms or a hydroxyalkyl having from 1 to 6 carbon atoms; R₅ is a long-chain aliphatic hydrocarbon having from 8 to 24 atoms; Y is a heteroatom selected from the group consisting of O, N and S; R₆ is an alkyl having from 1 to 8 carbon atoms; each R₇ is the same or different and is an alkyl having from 1 to 8 carbon atoms, hydroxyalkyl having from 1 to 8 carbon atoms or benzyl; X is an anion such as chloride, bromide, methosulfate, ethosulfate, tosylate, acetate, lactate, phosphate and nitrate. The alkyl substituents present in formula I and II above may be straight chained or branched and may be saturated or unsaturated, but not substituted.

The long-chain aliphatic C₈₋₂₄ hydrocarbon group of the substantially water insoluble quaternary ammonium compound of the present invention does not have to solely, or primarily, contain one chain length, i.e., the long chain need not be only lauryl (C₁₂), myristyl (C₁₄), stearyl (C₁₈) or behenyl (C₂₂). Rather, a quaternary ammonium compound wherein the long chain hydrocarbon group is a mixture of lengths can be used in the present invention. Such quaternary ammonium compounds are prepared conveniently from naturally-occurring or synthetic materials, such as tallow, coconut oil, palm oil, soya and the like, or from synthetically produced mixtures.

Illustrative examples of quaternary compounds that satisfy the above formulas and which are substantially water insoluble include, but are not limited to: dicetyldimonium chloride, dicetyldimonium bromide, dicetyldimonium tosylate, stearyl dimethyl benzyl ammonium chloride, palmitamidopropyltrimonium chloride, behenamidopropyl trimonium chloride, palmitamidopropyltrimonium bromide, cocamidopropyl ammonium chloride, dibehenyldimonium chloride, stearylamidopropyltrimonium methosulafate, tallowamidotrimonium chloride, soyamidopropyltrimonium chloride and canolamidopropyltrimonium methosulfate.

Of these substantially water insoluble quaternary ammonium salts, dicetyldimonium chloride (VARISOFT® 432 PPG), dibehenyldimonium chloride and palmitamidopropyltrimonium chloride (VARISOFT® PATC) are highly preferred in the present invention.

The oil or hydrophobic organic component employed in the present invention is any oil or fatty organic compound which is not water dispersible on its own. Such compounds are well known to those skilled in the art and may be selected from the group of fatty esters made from a C₈₋₂₄ acid and a C₁₋₈ alcohol, dialkyl esters, propoxylated alcohols, propoxylated fatty acids, mineral oils, mineral seal oils, silicon oils, petrolatums, aliphatic hydrocarbons, paraffinic hydrocarbons, napthalic hydrocarbons, oils, spirits and mixtures thereof.

Some illustrative examples of preferred oils and hydrophobic organic compounds that can be employed in the present invention include: KEMESTER® 5822 (isocetyl stearate), FINSOLVE® TN (C₁₂₋₁₅ alkylbenzoate), STARFOL® OS (octyldodecyl stearate), Cochin oil, mineral oil, VARONIC® APM (PPG-3 myristyl ether), VARONIC® APS (PPG-11 stearyl ether), isopropyl palmitate and other like fatty acid esters, Klearol (light mineral oil), C₁₄₋C₁₇ n-paraf fins (light mineral oil), Kaydol (medium weight mineral oil), C₁₈-C₂₀ n-paraf fins (medium mineral oil), C₂₀₋C₂₄ branched paraffins (heavy mineral oil) and Witco Carnation Oil (heavy mineral oil), palm oil, avocado oil and other like natural whole oils such as TEA tree oil, for example.

Dispersants that are employed in the present invention include any compound which is capable of preventing the oil or hydrophobic organic compound from coalescing into particle sizes that are larger than the wavelength of visible light. That is, the dispersant employed in the present invention does not permit the formation of a microemulsion in which the average particle size is above 100 nm. Instead, the dispersant employed in the present invention provides the coalescene of particle sizes that are less than the visible wavelength of light.

Suitable dispersants that can be employed in the present invention include, but are not limited to: hydroxypivalyl hydroxypivalate and its alkoxylated (1 EO) derivative, TMPD (trimethyl-1,3-pentanediol), TMPD ethoxylate (1 EO), 1,2 cyclohexanedimethanol, 1,4-cyclohexanedimethanol, isopentyldiol, 1,2-hexanediol, hexylene glycol, propylene glycol, isoprene glycol, sorbitan ethoxylates, 2-butoxyethanol, C₆-C₁₂ diols/triols and ester diols/triols and their alkoxylated derivatives, glycol ethers, and any mixtures and combinations thereof. It should be noted that propoxylated ethoxylated alcohols such as PPG-3-isosteareth-9 (ADOGEN® 66 PE-12) and ethoxylated alcohols such as C₁₁₋₅ Pareth-12 (TERGITOL® 15-S-12 surfactant) may also be employed as long as the same are used in conjunction with one of the above-identified dispersants.

Of the above mentioned dispersants, TMPD ethoxylate (1 EO), 1,2-hexanediol, hexylene glycol, propylene glycol and a mixture of hexylene glycol and a propoxylated ethoxylated alcohol, such as PPG-2-isosteareth (ADOGEN® 66 PE-12), are particularly preferred.

The weight ratio of component (a) to component (b) to compound (c) in the microemulsion of the present invention is from about 2:0.5:0.4 to about 0.5:2:0.8. More preferably, the weight ratio of component (a) to component (b) to compound (c) in the microemulsion is from about 0.8:1.2:0.4 to about 1.2:0.8:0.75. Most preferably the weight ratio of component (a) to component (b) to compound (c) in the microemulsion is from about 0.9:1.1:0.5 to about 1.1:0.9:0.6.

The microemulsion of the present invention is prepared by adding the above mentioned three components into a suitable reaction vessel containing at least a mixing means and then emulsifying the components under conditions that are capable of forming a microemulsion which have the properties mentioned above. That is, the emulsifying step is capable of forming a microemulsion in which the resultant emulsion is clear and has average particle sizes that are less than the wavelength of light, i.e., less than 100 nm.

The three components may be added in any order, using conventional processes well known to those skilled in the art. The microemulsion may be produced at room temperature or elevated temperatures up to 90° C. can be employed. The microemulsions of the present invention may be made by hand stirring or, if needed, by using a mechanical mixer.

The resultant microemulsion can also be diluted with water (50 weight % or more) to obtain a clear, dilutable microemulsion. Specifically, when a clear, diluted microemulsion is desired, from about 52 to about 98 weight % of water can be added to the microemulsion. More specifically, when a diluted microemulsion is desired from about 75 to about 95 weight % water can be employed, with a range of from about 80 to about 92 weight % being most highly preferred. The amount of water employed does not affect the weight ratio of components (a)-(c) present in the microemulsion.

It is also observed that upon dilution, the microemulsion of the present invention remains clear. Another characteristic of the diluted microemulsion is that at commercial concentrations it is freeze/thaw stable. This means that upon freezing and subsequent thawing the diluted microemulsion remains clear.

The above two properties are unique to the inventive microemulsion and provide a product which can be sold in either a diluted or undiluted form.

In addition to the above essential components, the present invention also contemplates the addition of other components that are typically employed in hair conditioner formulations or other personal care products. The additional components that can be employed in the present invention are conventional and are well known to those skilled in the art.

For example, when the microemulsion of the present invention is employed in a hair conditioner formulation, the formulation may also include: a personal care emollient such as acetylated lanolin, aminopropyl dimethicone and ammonium hydrolyzed collagen; a personal care emulsifier such as beheneth-5, beheneth-10, beheneth-20, butylglycoside caprate and cetearate-2; an anionic surfactant such as ammonium lauryl sulfate, sodium lauryl sulfate, ammonium laureth sulfate, sodium lauryl sulfate and the like; an amphoteric surfactant such as cocamidopropyl betaine, lauramidopropyl betaine and cocobetaine; an alkanolamide such as almondamide diethanolamine (DEA) behenamide DEA, cocamide DEA and lauramide DEA; an amine oxide such as behenamine oxide, cocamidopropylamine oxide and cocamide oxide; a perfume; a preservative; an antimicrobial preservative; an antistatic agent; a dye; a colorant; an extract; a lubricant or slip agent; a wetting agent and other like additives typically employed in hair conditioner formulations. One or more of the above-mentioned additives may be employed in the present invention.

The above-mentioned additives are employed with the microemulsion of the present invention in amounts that are typically employed in the field of personal care products.

The following examples are given to illustrate the scope of the present invention. Because these examples are given for illustrative purposes only, the invention embodied therein should not be limited thereto.

### EXAMPLE 1

The following formulations (Formulations A-G) were prepared using compounds and amounts that are within the scope of the present invention. The following formulations are clear in the concentrated form and remain clear even after diluting with 10% water. Moreover, Formulations A-G were also found to be clear after freeze/thawing. Unless otherwise specified, the freeze/thaw determination was carried out by storage in a conventional freezer and then thawing. This freeze/thaw cycle was repeated 3 times. The clarity determination was carried out by examination with the eye or with a UV/visible detector.

| Ingredient | Weight % |
|---|---|
| Concentrate Formulation A: | |
| VARISOFT ® 432 PPG (Dicetyldimoniun Chloride) | 18% |
| KEMESTER ® 5822 (Isocetyl Stearate, from Exal | 18% |
| 16 isocetyl alcohol) | |
| TMPD-1EO (Trimethyl-1,3-Pentanediol Ethoxylate) | 12% |
| Hexylene Glycol | 2% |
| DI Water | 50% |
| Concentrate Formulation B: | |
| VARISOFT ® PATC (Palmitamidopropyl | 16% |
| Trimonium Chloride) | |
| WITCONOL ® APM (PPG-3 Myristyl Ether) | 18% |
| TMPD-1EO | 12% |
| DI Water | 54% |
| Concentrate Formulation C: | |
| VARISOFT ® PATC | 16% |
| WITCONOL ® APM | 16% |
| 1,2 Hexane Diol | 12% |
| DI Water | 56% |
| Concentrate Formulation D: | |
| VARISOFT ® PATC | 16% |
| WITCONOL ® APM | 16% |
| TMPD-1EO | 12% |
| DI Water | 56% |
| Concentrate Formulation E: | |
| VARISOFT ® 432 | 18% |
| KEMESTER ® 5822 | 18% |
| TMPD-1EO | 12% |
| DI Water | 50% |
| Hexylene Glycol | 2% |
| Concentrate Formulation F: | |
| VARISOFT ® 432 | 18% |
| KEMESTER ® 5822 | 18% |
| TMPD-1EO | 12% |
| DI Water | 50% |
| Propylene Glycol | 2% |
| Concentrate Formulation G: | |
| VARISOFT ® 432 | 18% |
| KEMESTER ® 5822 | 18% |
| TMPD-1EO | 12% |
| DI Water | 50% |
| TERGITOL ® 15-S-12 Surfactant (C₁₁₋₁₅ Pareth 12 | 2% |
| from Union Carbide) | |

### COMPARATIVE EXAMPLES 1-2

The following formulations (CE 1-2) represent formulations that are outside the scope of the present invention inasmuch as the diluted versions were not freeze/thaw stable.

CE Formulation 1:

| Ingredient | Weight % |
|---|---|
| VARISOFT ® 432 PPG (Dicetyldimonium Chloride) | 25% |
| Klearol Mineral Oil (Mineral Oil) | 12.5% |
| TMPD-1EO (Trimethyl-1,3-Pentanediol Ethoxylate) | 50% |
| TERGITOL ® 15-S-12 | 12.5% |

Dilutions were made by mixing the above concentrate with water. 10% and 35% dilutions of this concentrate were cloudy. The 14% dilution was initially clear but not freeze/thaw stable.

CE Formulation 2:

| Ingredient | Weight % |
|---|---|
| VARISOFT ® 432 PPG (Dicetyldimonium Chloride) | 28.6% |
| Klearol Mineral Oil (Mineral Oil) | 14.3% |
| TMPD-1EO | 57.1% |

Dilutions were made by mixing the above concentrate with water. 10%, 12%, 20%, 24%, 25% and 35% dilutions of this concentrate were cloudy. The 14% dilution was initially clear but not freeze/thaw stable.

### EXAMPLE 2

The following formulations are within the scope of the present invention and the formulations are freeze/thaw stable upon dilution with water.

Formulation H:

| Ingredients | Weight % |
|---|---|
| VARISOFT ® 432 PPG (Dicetyldimonium Chloride) | 25.0 |
| TMPD-1EO | 50.0 |
| TERGITOL ® 15-S-12 | 12.5 |
| Klearol Mineral Oil | 12.5 |

The concentrate was made by combining the ingredients in order, then stirring until even. A serial dilution was then made with water. Dilutions containing 10, 12, 14, 16, 18, 20, 22, 24, 25, and 35% of the concentrate were made by adding the concentrate to the water and then stirring until even.

The 10% and 35% dilutions were cloudy after standing overnight. The 12% dilution cleared after five days. The following dilutions were clear after stirring and being allowed to stand overnight: 14, 16, 18, 20, 22, 24, and 25%. These dilutions were put through a freeze/thaw cycle overnight in a −4° C. freezer. After standing at room temperature for 8 hours, the 20, 22, 24 and 25% dilution were clear. After 24 hours of standing, the 16 and 18% dilutions were also clear.

The 16, 18, 20, 22, 24, and 25% solutions will go through a second round of freeze/thaw tests.

The following concentrate formulation was made:

Formulation I:

| Ingredients | Weight % |
|---|---|
| VARISOFT ® 432 PPG | 28.6 |
| TMPD-1EO | 57.1 |
| Klearol Mineral Oil | 14.3 |

The concentrate was made by combining the ingredients in order, then stirring until even. A serial dilution was then made with water. Dilutions containing 10, 12, 14, 16, 18, 20, 22, 24, 25, and 35% of the concentrate were made by adding the concentrate to the water and then stirring until even.

Of the dilutions, only the following were clear: 14, 16 and 18%. These dilutions were put through a freeze/thaw cycle overnight in a −4° C. freezer. After 8 hours of standing at room temperature, the 16 and 18% dilutions were clear. They will undergo another freeze/thaw test.

In the second freeze/thaw test for Formulation H, the samples were put in a freezer overnight at −4° C. After 8 hours at room temperature, the following dilutions were clear: 18%, 20%, 22%, 24%, and 25%. After standing overnight, the 18% dilution was clear.

In the second freeze/thaw test for Formulation I, the samples were put in a freezer overnight at −4° C. After 8 hours at room temperature, the 18% dilution was clear. After standing overnight, the 16% dilution was clear.

In the third freeze/thaw test for Formulation H, the samples were put in a freezer overnight at −4° C. After 8 hours at room temperature, the following dilutions were clear: 18%, 20%, 22%, 24%, and 25%. After standing overnight, the 18% dilution was clear.

In the third freeze/thaw test for Formulation I, the samples wer put in a freezer overnight at −4° C. After 8 hours at room temperature, the 18% dilution was clear. After standing overnight, the 16% dilution was clear.

During each of the three thaw cycles for both products, the samples were shaken slightly two or three times to judge clarity and to remix the formulations.

### COMPARATIVE EXAMPLE 3

The following prior art microemulsion was prepared using the ingredients and amounts specified below.

| | | |
|---|---|---|
| HMDA (Hexamethylenediamine alkoxylated | 15 | g |
| ester diamine) + 2 moles tallow fatty acid | | |
| Isopropyl alcohol (IPA) | 2.6 | g |
| TMPD/CHDM (80/20) | 11.7 | g |
| Methyl Oleate | 18 | g |
| DI Water | 52.7 | |

The HMDA, IPA, and Methyl Oleate were weighed into a beaker and stirred until uniform.

16 g of TMPD and 4 g of CHDM (cyclohexanedimethanol) were weighed into a separate beaker and melted until uniform liquid. 11.7 g of this mixture was added to the HMDA, IPA, methyl oleate blend and mixed until uniform.

52.7 g of DI water were added while mixing. The result was a clear yellow solution. 10 g of solution was diluted in 90 ml of DI water. The result was a off-white cloudy solution. 5 g of mixture was diluted with 95 ml of H₂O resulting in an off-white cloudy solution.

Transmittance measurements were done at 460 nm:

| | |
|---|---|
| DI water | 100% |
| 50% solution | 80% |
| 10% solution | 13% |
| 5% solution | 31% |

The above prior art formulation was not clear.

### EXAMPLE 3

Three formulations J, K and L were made using compounds and amounts that are within the scope of the present invention. Formulation J comprises VARISOFT® PATC, as the quat, WITCONOL® APM, as the oil, and propylene glycol, as the dispersant. Formulation K comprises VARISOFT® 432 PPG, Klearol and hexylene glycol, while Formulation L comprises VARISOFT® 432 PPG, a light mineral oil and, as the dispersant, a mixture of 2 parts hexylene glycol and 1 part ADOGEN® 66 PE-12.

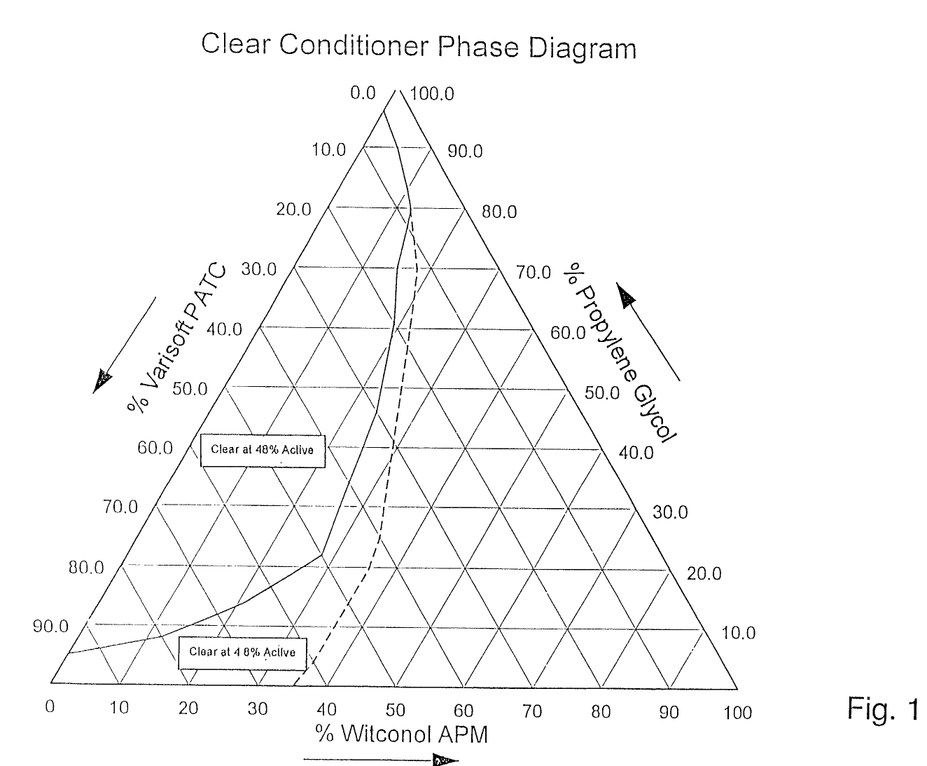

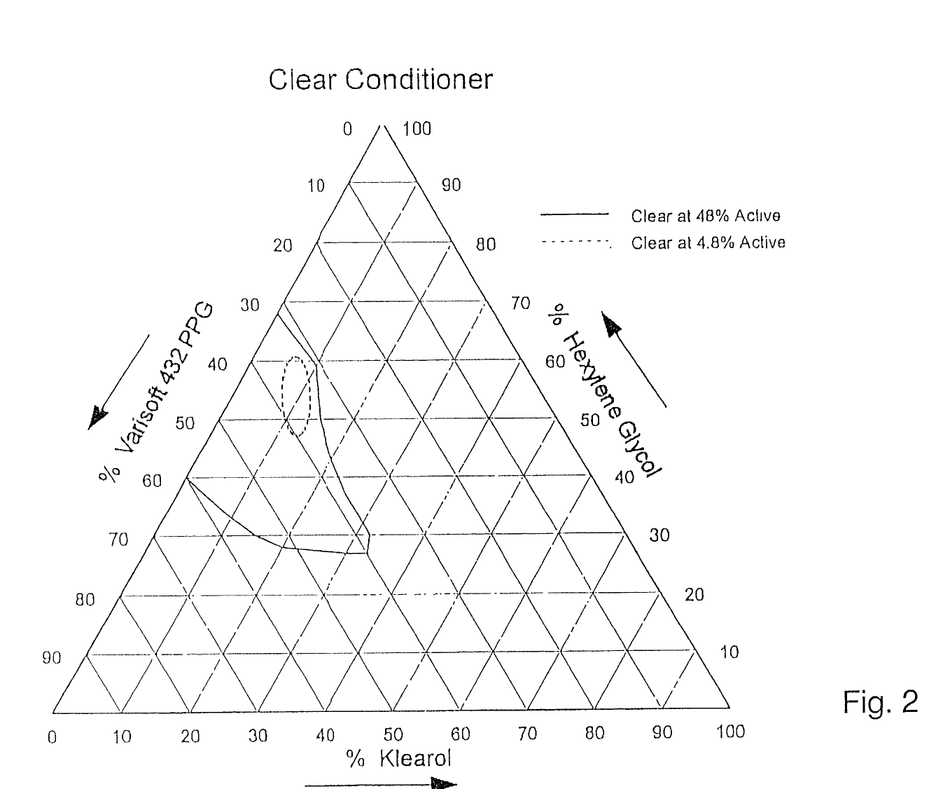

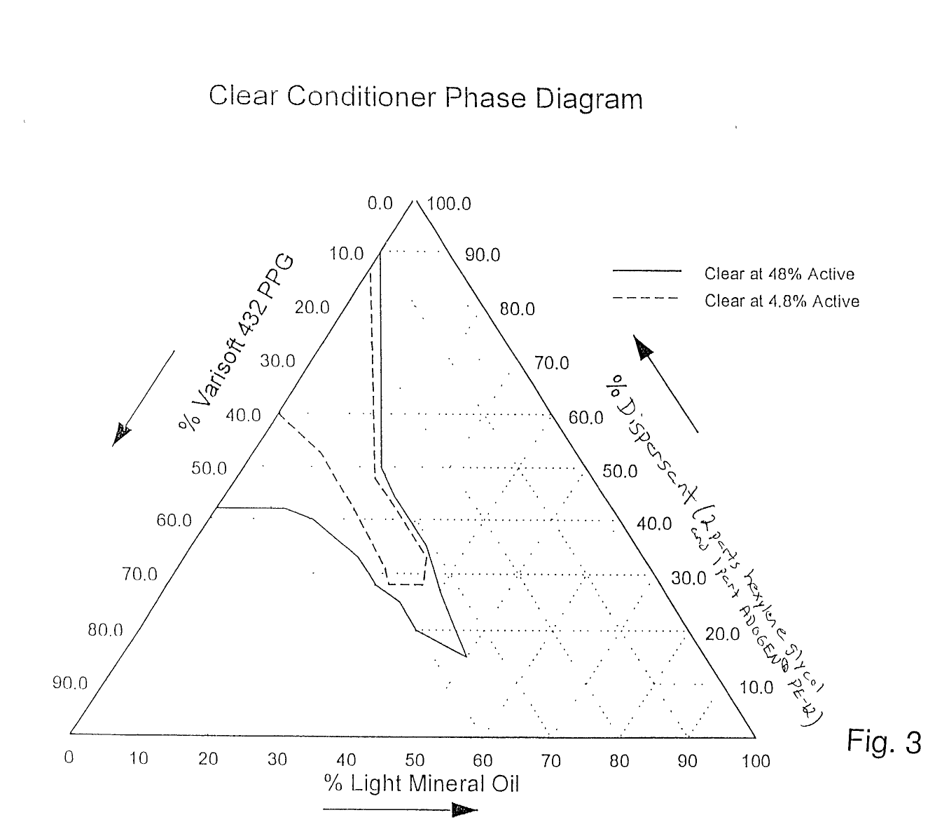

Ternary phase diagrams shown in FIGS. 1-3 were constructed in the following manner (It is noted that FIG. 1 represents the ternary phase diagram for Formulation J; FIG. 2 represents the ternary phase diagram for Formulation K; and FIG. 3 is the ternary phase diagram for Formulation L):

(1) Varying weight ratios of quat, oil and dispersant totaling 24 grams were weighed into a beaker and stirred by hand with a plastic rod.

(2) 26 grams water was added to the beaker and the contents stirred by hand. This emulsion was considered 48% active and was judged to be clear or hazy in appearance.

(3) 5 grams of the 48% active emulsion was diluted with 45 ml water and stirred by hand. The resulting emulsion was considered 4.8% active and judged to be clear or hazy in appearance.

(4) The weighted percent of the quat, oil and dispersant before water addition was plotted on the phase diagram as clear or hazy depending on the condition of the resulting emulsion.

(5) The area of the phase diagram depicting clear emulsion was outlined in the final phase diagram shown in FIGS. 1-3.

The ternary phase diagrams of FIGS. 1-3 show the regions where the formulations are clear at 48% active and 4.8% active. Formulations within these regions are representative of the present invention, while formulations outside the regions are outside the scope of the present invention.

### EXAMPLE 4

This example was conducted to show that the method of dilution of a clear microemulsion of the present invention has an effect on the final clarity of the diluted microemulsion. Specifically, a clear microemulsion (prior to dilution) was made in accordance with the present invention by using the following components and concentrations.

| Component | Grams |
|---|---|
| VARISOFT ® 432 PPG | 16.8 |
| Klearol Oil | 4.8 |
| Hexylene Glycol | 26.4 |
| Water | 52.0 |

The above prepared microemulsion was clear and evidenced a % Transmittance at 460 nm of 99.2.

In one experiment, the above microemulsion was diluted by adding water in small increments while stirring. The following transmittance data (% T at 460 nm) was observed at the various increments of water added.

| ml H₂O total | % T at 460 nm |
|---|---|
| 0 | 99.2 |
| 5 | 96.0 |
| 10 | 2.1 |
| 15 | 2.3 |
| 20 | 2.2 |
| 25 | 2.1 |
| 30 | 2.1 |
| 40 | 2.3 |
| 50 | 3.0 |
| 60 | 4.3 |
| 70 | 5.7 |
| 80 | 7.2 |
| 90 | 8.8 |
| 110 | 12.3 |
| 135 | 16.4 |

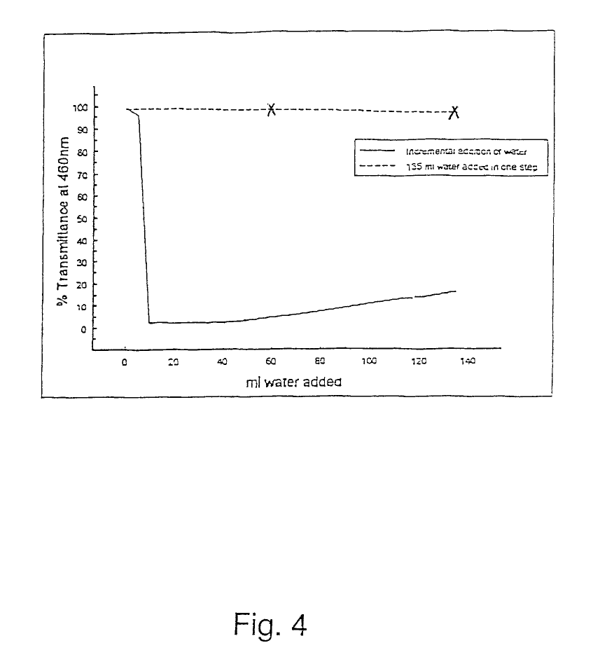

In a separate experiment, the entire 135 ml of water was added to the microemulsion at the same rate. Using this method of dilution, the % T was 96.6. The data for the two experiments are plotted in FIG. 4. The dotted. line represents the method of the present invention, while the solid line represents incremental addition of water.

### EXAMPLE 5

In this example, a clear conditioner formulation. of the present invention was prepared. The same formulation was made up using Kaydol oil in place of Klearol oil. Also the same formulation was made using the 100% active version of the quaternary ammonium compound (quat) and adding the dispersant (propylene glycol) to the formulation. These samples were tested for transmittance at 460 nm and then diluted to 10% in water and tested for transmittance.

| | Standard | Kaydol Std. | Quat form |
|---|---|---|---|
| Dicetyldimonium | 16.8% | 16.8% | — |
| Chloride | | | |
| Quaternium 18 | — | — | 10.1 |
| (100%) | | | |
| Klearol (low | 4.8 | — | 4.8 |
| density, light | | | |
| mineral oil) | | | |
| Kaydol (high | — | 4.8 | — |
| density mineral | | | |
| oil) | | | |
| Hexylene Glycol | 26.4 | 26.4 | 26.4 |
| Propylene Glycol | — | — | 6.7 |
| Water | 52 | 52 | 52 |
| % T | 99 | 26.8* | 23.2* |
| % T (10% in water) | 95.8 | 51.6* | 96.6 |

*Sample split into two phases on sitting overnight. The type of oil and the solvent used for the quat makes a difference in the final clarity of the clear conditioner.

### EXAMPLE 6

This example demonstrates that Quaternium 18 and Quaternium 81 do not form stable microemulsions with mineral oils and hexylene glycol or propylene glycol.

The following formulations were prepared (% wt/wt shown) to demonstrate that these particular combinations of cationics and mineral oil (regardless of type or weight) do not form clear, dilutable microemulsions. These formulations are concentrates.

| | A | B | C | D | E | F | G | H | I | J | K | L |
|---|---|---|---|---|---|---|---|---|---|---|---|---|
| Quaternium | 18 | 18 | 18 | 18 | 18 | 18 | | | | | | |
| 18 | | | | | | | | | | | | |
| Quaternium | | | | | | | 18 | 18 | 18 | 18 | 18 | 18 |
| 81 | | | | | | | | | | | | |
| Klearol | 18 | | | 18 | | | 18 | | | 18 | | |
| Kaydol | | 18 | | | 18 | | | 18 | | | 18 | |
| Carnation | | | 18 | | | 18 | | | 18 | | | 18 |
| Oil | | | | | | | | | | | | |
| Hexylene | 12 | 12 | 12 | | | | 12 | 12 | 12 | | | |
| Glycol | | | | | | | | | | | | |
| Propylene | | | | 12 | 12 | 12 | | | | 12 | 12 | 12 |
| Glycol | | | | | | | | | | | | |
| Water | 52 | 52 | 52 | 52 | 52 | 52 | 52 | 52 | 52 | 52 | 52 | 52 |

Each concentrate was diluted 1 in 10 to reach a level of 1.8% cationic (Quaternium 18, for instance). None of these formulations or their dilutions were clear at room temperature.

### EXAMPLE 7

In this example, several formulations from U.S. Pat. No. 4,454,049 were made and tested for clarity at the 10% dilution level.

| Patent Example # | 6 | 7 | 9 | 11 | 12 |
|---|---|---|---|---|---|
| Quaternium 18 | 27% | 50% | — | — | 37% |
| (dimethyldi (hydrogenated | | | | | |
| tallow) ammonium chloride) | | | | | |
| Quaternium 81 | — | — | 20 | 30 | — |
| (3-methyl-1-(2-oleyamido)ethyl- | | | | | |
| 2-oleyl-imidazolinium | | | | | |
| methosulphate | | | | | |
| Klearol | — | — | — | 14 | — |
| Carnation Oil | 53 | 25 | 3 | — | 17 |
| Hexylene Glycol | 18 | — | 27 | 15 | 17 |
| Propylene Glycol | — | 25 | — | — | — |
| Polystyrene Latex | 1 | — | — | — | 1 |
| Water | — | — | 50 | 40 | 27 |

Each formulation was tested for % Transmittance at 460 nm. Dilutions of 10% formulation in water were tested for %T.

| Sample # | % T Formulation | % T 10% in water |
|---|---|---|
| 6 | 43 | 0 |
| 7 | 41.4 | 0 |
| 9 | 73.5 | 0.3 |
| 11 | 33.7 | 0.1 |
| 12 | 1.7 | 0 |

None of these formulations were clear at 10% dilution level.

While the present invention has been particularly shown and described with respect to preferred embodiments thereof, it will be understood by those skilled in the art that the foregoing and other changes in form and detail may be made without departing from the spirit and scope of the present invention. It is therefore intended that the present invention not be limited to the exact forms and details described and illustrated, but fall within the appended claims.

What is claimed is:

1. A clear microemulsion comprising:
(a) a substantially water insoluble quaternary ammonium salt containing at least one long-chain aliphatic C₈₋₂₄ hydrocarbon group;
(b) an oil or hydrophobic organic compound that is not dispersible in water on its own; and
(c) a dispersant capable of preventing component (b) from coalescing into particle sizes that are above the wavelength of light, wherein the weight ratio of component (a) to component (b) to component (c) is from about 2:0.5:0.4 to about 0.5:2:0.8.

2. The clear microemulsion of claim 1 wherein said substantially water insoluble quaternary ammonium salt is a compound having one of the following formulas: 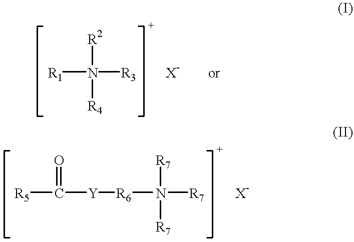
wherein R₁ is a long-chain aliphatic hydrocarbon having from 8 to 24 carbon atoms; R₂ is a long-chain aliphatic hydrocarbon having from 8 to 24 carbon atoms, an alkyl group having from 1 to 6 carbon atoms or a hydroxyalkyl having from 1 to 6 carbon atoms; R₃ is hydrogen, benzyl, an alkyl having from 1 to 6 carbon atoms or a hydroxyalkyl having from 1 to 6 carbon atoms; R₄ is an alkyl having from 1 to 6 carbon atoms or a hydroxyalkyl having from 1 to 6 carbon atoms; R₅ is a long-chain aliphatic hydrocarbon having from 8 to 24 atoms; Y is a heteroatom selected from the group consisting of O, N and S; R₆ is an alkyl having from 1 to 8 carbon atoms; each R₇ is the same or different and is an alkyl having from 1 to 8 carbon atoms, hydroxyalkyl having from 1 to 8 carbon atoms or benzyl.

3. The clear microemulsion of claim 1 wherein said long-chain aliphatic C₈-C₂₄ hydrocarbon group of said substantially water insoluble quaternary ammonium compound has a mixture of chain lengths.

4. The clear microemulsion of claim 3 wherein said substantially water insoluble quaternary ammonium compound having a mixture of long-chain lengths is prepared from a naturally-occurring material selected from the group consisting of tallow, coconut oil, palm oil and soya.

5. The clear microemulsion of claim 4 wherein soya is the naturally-occurring material.

6. The clear microemulsion of claim 2 wherein said substantially water insoluble quaternary ammonium compound having a mixture of chain lengths is prepared from a synthetic material.

7. The clear microemulsion of claim 2 wherein said substantially water insoluble quaternary ammonium compound is selected from the group consisting of dicetyldimonium chloride, dicetyldimonium bromide, dicetyldimonium tosylate, stearyl dimethyl benzyl ammonium chloride, palmitamidopropyltrimonium chloride, behenamidopropyl trimonium chloride, palmitamidopropyltrimonium bromide, cocamidopropyl ammonium chloride, dibehenyldimonium chloride, stearylamidopropyltrimonium methosulafate, tallowamidotrimonium chloride, soyamidopropyltrimonium chloride and canolamidopropyltrimonium methosulfate.

8. The clear microemulsion of claim 7 wherein said quaternary ammonium compound is dicetyldimonium chloride, dibehenyldimonium chloride or palmitamidopropyltrimonium chloride.

9. The clear microemulsion of claim 1 wherein compound (b) is selected from the group consisting of fatty esters made from a C₈₋₂₄ acid and a C₁₋₈ alcohol, dialkyl esters, propoxylated alcohols, propoxylated fatty acids, mineral oils, mineral seal oils, silicon oils, petrolatums, monoglycerides, diglycerides, triglycerides, aliphatic hydrocarbons, paraffinic hydrocarbons, napthalic hydrocarbons, oils, spirits and mixtures thereof.

10. The clear microemulsion of claim 9 wherein compound (b) is a mineral oil, PPG-3 myristyl ether or a paraffinic hydrocarbon.

11. The clear microemulsion of claim 1 wherein said dispersant is selected from the group consisting of hydroxypivalyl hydroxypivalate and its alkoxylated derivative, TMPD (trimethyl-1,3-pentanediol), TMPD ethoxylate (1 EO), 1,2 cyclohexanedimethanol, 1,4-cyclohexanedimethanol, isopentyldiol, 1,2-hexanediol, hexylene glycol, propylene glycol, isoprene glycol, 2-butoxyethanol, C₆-C₁₂ diols/triols and ester diols/triols and their alkoxylated derivatives, glycol ethers, and any mixtures and combinations thereof.

12. The clear microemulsion of claim 11 wherein said dispersant is TMPD ethoxylate (1 EO), 1,2-hexane diol, hexylene glycol, propylene glycol or a mixture of hexylene glycol and PPG-3-isosteareth-9.

13. The clear microemulsion of claim 1 wherein said weight ratio of component a:b:c is from about 0.8:1.2:0.4 to about 1.2:0.8:0.75.

14. The clear microemulsion of claim 13 wherein said weight ratio of component a:b:c is from about 0.9:1.1:0.5 to about 1.1:0.9:0.6.

15. The clear microemulsion of claim 1 wherein said substantially water insoluble quaternary ammonium compound is palmitamidopropyltrimonium chloride, said component (b) is PPG-3 myristyl ether and said dispersant is propylene glycol.

16. The clear microemulsion of claim 1 wherein said substantially water insoluble quaternary ammonium compound is palmitamidopropyltrimonium chloride, said component (b) is PPG-3 myristyl ether and said dispersant is TMPD ethoxylate (1 EO).

17. The clear microemulsion of claim 1 wherein said substantially water insoluble quaternary ammonium compound is dicetyldimonium chloride, said component (b) is a light mineral oil and said dispersant is hexylene glycol.

18. The clear microemulsion of claim 1 wherein said substantially water insoluble quaternary ammonium compound is dicetyldimonium chloride, said component (b) is a light mineral oil and said dispersant is TMPD ethoxylate (1 EO).

19. The clear microemulsion of claim 1 wherein said quaternary ammonium compound is dicetyldimonium chloride, said component (b) is a light mineral oil and said dispersant is a mixture of hexylene glycol and PPG-3-isosteareth-9.

20. The clear microemulsion of claim 1 wherein said microemulsion has average particle sizes of less than 100 nm.

21. A substantially dilutable microemulsion which remains clear after the addition of water, said infinitely dilutable microemulsion comprising:
(a) a substantially water-insoluble quaternary ammonium salt containing at least one long-chain aliphatic C₈₋₂₄ hydrocarbon group;
(b) an oil or hydrophobic organic compound that is not dispersible in water on its own; and
(c) a dispersant capable of preventing component (b) from coalescing into particle sizes that are above the wavelength of light, wherein the weight ratio of component (a) to component (b) to component (c) is from 2:0.5:0.4 to about 0.5:2:0.8; and
(d) at least 50% water.

22. The substantially dilutable microemulsion of claim 21 wherein from about 52 to about 98 weight % water is employed.

23. The substantially dilutable microemulsion of claim 21 wherein said substantially water insoluble quaternary ammonium salt is a compound having one of the following formulas: 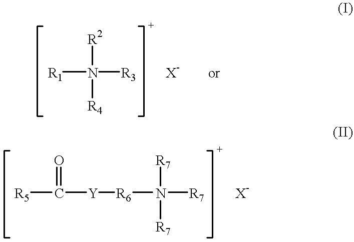
wherein R₁ is a long-chain aliphatic hydrocarbon having from 8 to 24 carbon atoms; R₂ is a long-chain aliphatic hydrocarbon having from 8 to 24 carbon atoms, an alkyl group having from 1 to 6 carbon atoms or a hydroxyalkyl having from 1 to 6 carbon atoms; R₃ is hydrogen, benzyl, an alkyl having from 1 to 6 carbon atoms or a hydroxyalkyl having from 1 to 6 carbon atoms; R₄ is an alkyl having from 1 to 6 carbon atoms or a hydroxyalkyl having from 1 to 6 carbon atoms; R₅ is a long-chain aliphatic hydrocarbon having from 8 to 24 atoms; Y is a heteroatom selected from the group consisting of O, N and S; R₆ is an alkyl having from 1 to 8 carbon atoms; each R₇ is the same or different and is an alkyl having from 1 to 8 carbon atoms, hydroxyalkyl having from 1 to 8 carbon atoms or benzyl.

24. The substantially dilutable microemulsion of claim 21 wherein said long-chain aliphatic C₈-C₂₄ hydrocarbon group of said substantially water insoluble quaternary ammonium compound has a mixture of chain lengths.

25. The substantially dilutable microemulsion of claim 24 wherein said substantially water insoluble quaternary ammonium compound having a mixture of long-chain lengths is prepared from a naturally-occurring material selected from the group consisting of tallow, coconut oil, palm oil and soya.

26. The substantially dilutable microemulsion of claim 25 wherein soya is the naturally-occurring material.

27. The substantially dilutable microemulsion of claim 24 wherein said substantially water insoluble quaternary ammonium compound having a mixture of chain lengths is prepared from a synthetic material.

28. The substantially dilutable microemulsion of claim 23 wherein said substantially water insoluble quaternary ammonium compound is selected from the group consisting of dicetyldimonium chloride, dicetyldimonium bromide, dicetyldimonium tosylate, stearyl dimethyl benzyl ammonium chloride, palmitamidopropyltrimonium chloride, behenamidopropyl trimonium chloride, palmitamidopropyltrimonium bromide, cocamidopropyl ammonium chloride, dibehenyldimonium chloride, stearylamidopropyltrimonium methosulafate, tallowamidotrimonium chloride, soyamidopropyltrimonium chloride and canolamidopropyltrimonium methosulfate.

29. The substantially dilutable microemulsion of claim 28 wherein said substantially water insoluble quaternary ammonium compounds is dicetyldimonium chloride, dibehenyldimonium chloride or palmitamidopropyltrimonium chloride.

30. The substantially dilutable microemulsion of claim 21 wherein compound (b) is selected from the group consisting of fatty esters made from a C₈₋₂₄ acid and a C₁₋₈ alcohol, dialkyl esters, propoxylated alcohols, propoxylated fatty acids, mineral oils, mineral seal oils, silicon oils, petrolatums, monoglycerides, diglycerides, triglycerides, aliphatic hydrocarbons, paraffinic hydrocarbons, napthalic hydrocarbons, oils, spirits and mixtures thereof.

31. The substantially dilutable microemulsion of claim 30 wherein compound (b) is a mineral oil, PPG-3 myristyl ether or a paraffinic hydrocarbon.

32. The substantially dilutable microemulsion of claim 21 wherein said dispersant is selected from the group consisting of hydroxypivalyl hydroxypivalate and its. alkoxylated derivative, TMPD (trimethyl-1,3-pentanediol), TMPD ethoxylate (1 EO), 1,2 cyclohexanedimethanol, 1,4-cyclohexanedimethanol, isopentyldiol, 1,2-hexanediol, hexylene glycol, propylene glycol, isoprene glycol, 2-butoxyethanol, C₆-C₁₂ diols/triols and ester diols/triols and their alkoxylated derivatives, glycol ethers, and any mixtures and combinations thereof.

33. The substantially dilutable microemulsion of claim 32 wherein said dispersant is TMPD ethoxylate (1 EO), 1,2-hexane diol, hexylene glycol, propylene glycol or a mixture of hexylene glycol and PPG-3-isosteareth-9.

34. The substantially dilutable microemulsion of claim 21 wherein said weight ratio of component a:b:c is from about 0.8:1.2:0.4 to about 1.2:0.8:0.75.

35. The substantially dilutable microemulsion of claim 34 wherein said weight ratio of component a:b:c is from about 0.9:1.1:0.5 to about 1.1:0.9:0.6.

36. The substantially dilutable microemulsion of claim 21 wherein said substantially water insoluble quaternary ammonium compound is palmitamidopropyltrimonium chloride, said component (b) is PPG-3 myristyl ether and said dispersant is propylene glycol.

37. The substantially dilutable microemulsion of claim 21 wherein said substantially water insoluble quaternary ammonium compound is palmitamidopropyltrimonium chloride, said component (b) is PPG-3 myristyl ether and said dispersant is TMPD ethoxylate (1 EO).

38. The substantially dilutable microemulsion of claim 21 wherein said substantially water insoluble quaternary ammonium compound is dicetyldimonium chloride, said component (b) is a light mineral oil and said dispersant is hexylene glycol.

39. The substantially dilutable microemulsion of claim 21 wherein said substantially water insoluble quaternary ammonium compound is dicetyldimonium chloride, said component (b) is a light mineral oil and said dispersant is TMPD ethoxylate (1 EO).

40. The substantially dilutable microemulsion of claim 21 wherein said substantially water insoluble quaternary ammonium compound is dicetyldimonium chloride, said component (b) is a light mineral oil and said dispersant is a mixture of hexylene glycol and PPG-3-isosteareth-9.

41. A method of making a clear dilutable microemulsion comprising the steps of:
(i) providing a clear microemulsion, said clear microemulsion comprising (a) a substantially water insoluble quaternary ammonium salt containing at least one long-chain aliphatic C₈₋₂₄ hydrocarbon group (b) an oil or hydrophobic organic compound that is not dispersible in water on its own; and (c) a dispersant capable of preventing component (b) from coalescing into particle sizes that are above the wavelength of light, wherein the weight ratio of component (a) to component (b) to component (c) is from 2:0.5:0.4 to about 0.5:2:0.8; and
(ii) quenching the clear microemulsion by adding a sufficient amount of water to provide a clear, diluted microemulsion.

42. The method of claim 41 wherein from about 50 weight % or more of water is employed in step (ii).

43. The method of claim 42 wherein from about 52 to about 98 weight % water is employed in step (ii).
